(19) United States
(12) Patent Application Publication
(10) Pub. No.: US 2026/0355283 A1
(43) Pub. Date: Nov. 12, 2026

(54) METHOD FOR MASS-PRODUCING RAT OVA BY MEANS OF NOVEL SUPEROVULATION INDUCTION TREATMENT

(71) Applicant: NATIONAL UNIVERSITY CORPORATION KUMAMOTO UNIVERSITY, Kumamoto (JP)

(72) Inventors: Naomi Nakagata, Kumamoto (JP); Toru Takeo, Kumamoto (JP); Nobuyuki Mikoda, Saga (JP)

(73) Assignee: NATIONAL UNIVERSITY CORPORATION KUMAMOTO UNIVERSITY, Kumamoto (JP)

(21) Appl. No.: 19/477,061

(22) PCT Filed: Apr. 19, 2024
(86) PCT No.: PCT/JP2024/015563

§ 371 Date: Oct. 20, 2025

(30) Foreign Application Priority Data
Apr. 21, 2023 (JP) 2023-070207

(51) Int. Cl. A61K 38/24; A61K 39/395; A61P 15/08; C12N 5/073

(52) U.S. Cl. CPC A61K 38/24; A61K 39/3955; A61P 15/08; C12N 5/0604

(57) ABSTRACT

The present invention aims to provide a method for inducing superovulation in rats.

According to the present invention, there is provided a method for inducing superovulation characterized by simultaneously administering an anti-inhibin antibody and equine chorionic gonadotropin (eCG) to a female rat less than six weeks of age, followed by administration of human chorionic gonadotropin (hCG). The present invention also provides an in vitro fertilization method using oocytes prepared by the method of the present invention. The method of the present invention is applicable to various rat strains.

### TECHNICAL FIELD

The present invention relates to a method for inducing superovulation in female rats.

### BACKGROUND ART

Laboratory animals are essential for research to gain a deeper understanding of healthy and pathological conditions and to evaluate the efficacy of candidate therapeutics aimed at application to human disease. Comprehensive database platforms for laboratory rats have been developed, such as the National Bio Resource Project “Rat” in Japan and the Rat Genome Database in the United States. Rats have made significant contributions to the development of therapeutic drugs and regimens as models of human diseases, but their use in genetic modification research has not progressed as much as that of mice due to the difficulty of performing basic developmental engineering techniques such as superovulation and in vitro fertilization (IVF).

There are far fewer strains of rats than mice. Rats are larger than mice, which has the advantage that it is easier to collect blood, urine, and other samples over time and in larger quantities than mice. Rats also have the advantage that their organs and tissues are large enough to be used in surgical procedures. Furthermore, rats are characteristic in that they are docile and easily accustomed to humans, making them suitable for experiments such as behavioral observation.

Female rats reach sexual maturity between 50 and 80 days of age. The vaginal opening of females is closed with skin until about 20 days of age, but opens as they grow and their ovarian function develops. The optimum breeding period for females is between 60 and 80 days of age, where they ovulate 12 to 15 eggs in a 4-day cycle.

Superovulation is one of the most important techniques for effectively producing genetically modified rats. Superovulation methods using pregnant mare serum gonadotropin (PMSG; also known as equine chorionic gonadotropin (eCG)) and human chorionic gonadotropin (hCG) have been reported. However, some strains do not respond to such methods. Superovulation using PMSG and hCG has been reported to produce 17.0 ovulations per rat in the Wistar strain, 31.0 in the F344 strain, and 2.2 in the BN strain (Non-Patent Document 1).

Therefore, various methods have been proposed. For example, a method has been proposed in which ovaries collected from females administered PMSG are cultured in vitro, and then oocyte-granulosa cell complexes (OGCs) are collected. It has been reported that the number of OGCs collected per rat increased to 27.7 in the Wistar strain, 34.7 in the F344 strain, and 24.7 in the BN strain (Non-Patent Document 1). It has also been reported that 19.9 oocytes per rat were collected from the BN strain by treating rats with anti-inhibin serum (AIS) simultaneously with PMSG and hCG (Non-Patent Document 2). In this study, it was reported that by synchronizing the estrous cycle with luteinizing hormone-releasing hormone (LH-RH) and then administering PMSG and hCG, 22.3 oocytes were collected from BN rats, and furthermore, by synchronizing the estrous cycle with LHRH and AIS and then administering PMSG and hCG, 42.0 oocytes were collected from BN rats, 42.5 from Wistar, 33.3 from F344, 45.5 from LE, and 53.9 from SD rats per rat (Non-Patent Document 2).

### PRIOR ART DOCUMENTS

### Non-Patent Documents

Non-Patent Document 1: H. Taketsuru and T. Kaneko, Journal of Reproduction and Development, Vol. 62, No 5, 2016
Non-Patent Document 2: A. Honda et al., Scientific Reports (2019) 9:11571
Non-Patent Document 3: T. Aoto et al., Transgenic Res. (2011) 20:1245-12526
Non-Patent Document 4: N. Nakagata et al., Sci Rep. 2020 Jan. 9; 10(1):93. doi: 10.1038/s41598-019-57090-7
Non-Patent Document 5: K. Yamaga et al., Sci Rep. 2021; 11: 22641. doi: 10.1038/s41598-021-02166-6
Non-Patent Document 6: H. Kishi et al., The Journal of endocrinology 151(1): 65-75, 1996
Non-Patent Document 7: Nakagata, Exp. Anim. (Experimental Animals) 41(3), 387-388 (1992)

### SUMMARY OF THE INVENTION

### Technical Problem

As mentioned above, known techniques for superovulation in rats include administering pregnant mare serum gonadotropin (PMSG) and human chorionic gonadotropin (hCG), and further using luteinizing hormone-releasing hormone (LHRH) and anti-inhibin serum (AIS). However, to date, the only method that has been able to collect approximately 50 oocytes per rat from multiple strains of rats is the report by Honda et al. (Non-Patent Document 2) that uses LHRH, AIS, PMSG, and hCG. Since LHRH is expensive and cannot be stored for long periods of time, there has been a need for a simple method that uses a hormone reagent that can be stored for long periods of time and that can collect sufficient number of oocytes from various rat strains.

### Solution to Problem

As a result of intensive research to solve the problems, the present inventors have found that the effect of the hormone administered in superovulation treatment varies depending on the rat's age in weeks, and have completed the present invention. Specifically, it has been found that a sufficient superovulation can be induced by the combination of the hormone used and the age in weeks of the rat to which it is administered.

The present inventors also found that the quality of the oocytes produced by the superovulation treatment of the present invention was good, as confirmed by in vitro fertilization (IVF) with sperm. Furthermore, the developmental ability of the resulting embryos was confirmed by embryo transfer. As a result, the developmental ability was also good.

The present invention includes the following.
[1]A method for inducing superovulation, characterized by simultaneously administering an anti-inhibin antibody (preferably inhibin antiserum) and equine chorionic gonadotropin (eCG) to a female rat less than 6 weeks of age (preferably 3 weeks of age or older and less than 6 weeks of age), and subsequently administering human chorionic gonadotropin (hCG).
[2] The method according to [1], wherein the anti-inhibin antibody is administered as inhibin antiserum.
[3] The method according to [1] or [2], wherein the female rat is a female rat of 3 to 5 weeks of age.
[4] The method according to any one of [1] to [3], wherein the female rat is a female rat that has not been administered exogenous luteinizing hormone-releasing hormone (LH-RH) prior to administration of equine chorionic gonadotropin.
[5] The method according to any one of [1] to [3], wherein the female rat is a female rat that has not been administered exogenous gonadotropin-releasing hormone prior to administration of equine chorionic gonadotropin.
[6] The method according to any one of [2] to [5], wherein the inhibin antiserum to be administered is 0.3 mL or less per rat.
[7] The method according to [6], wherein the inhibin antiserum to be administered is 0.05 mL or more and 0.2 mL or less per rat.
[8] The method according to any one of [1] to [7], wherein the equine chorionic gonadotropin to be administered is 5 IU or more and 40 IU or less per rat (preferably 10 IU or more and 30 IU or less per rat).
[9] The method according to any one of [1] to [8], wherein the female rat is a female rat of a strain selected from the group consisting of CD (SD), Wistar, Long-Evans, F344, BN and LEW.
[10] An in vitro fertilization method comprising the steps of:
   a. producing oocytes using the method according to any one of [1] to [9]; and
   b. fertilizing the oocytes produced in the above step with sperm from a male rat (preferably sperm from a genetically modified male rat).
[11] An in vitro fertilization method comprising the steps of:
   (1) inducing superovulation by simultaneously administering an anti-inhibin antibody and equine chorionic gonadotropin (eCG) to a female rat less than 6 weeks of age (preferably 3 weeks of age or older and less than 6 weeks of age), and subsequently administering human chorionic gonadotropin (hCG);
   (2) collecting oocytes from the superovulated female rat; and
   (3) fertilizing the oocytes collected in the above step with sperm from a male rat (preferably sperm from a genetically modified male rat).
[12] The in vitro fertilization method according to [11], wherein the female rat is sperm derived from a male rat of a strain selected from the group consisting of CD (SD), Wistar, Long-Evans, F344, BN, and LEW.
[13] The method according to [11] or [12], wherein the anti-inhibin antibody is administered as inhibin antiserum.
[14] The method according to any one of [11] to [14], wherein the female rat is a female rat of 3 to 5 weeks of age.
[15] The method according to any one of [11] to [14], wherein the female rat is a female rat that has not been administered exogenous gonadotropin-releasing hormone prior to administration of equine chorionic gonadotropin.
[16] The method according to any one of [11] to [15], wherein the female rat is a female rat that has not been administered exogenous luteinizing hormone-releasing hormone (LH-RH) prior to administration of equine chorionic gonadotropin.
[17] The method according to any one of [11] to [16], wherein the inhibin antiserum to be administrated is 0.3 mL or less.
[18] The method according to [17], wherein the inhibin antiserum to be administrated is 0.05 mL or more and 0.2 mL or less.
[19] The method according to any one of [11] to [18], wherein the equine chorionic gonadotropin to be administrated is 5 IU or more and 40 IU or less (preferably 10 IU or more and 30 IU or less).

### Advantageous Effect of the Invention

The novel superovulation technique of the present invention is more efficient than conventional methods and can produce a significantly larger number of oocytes from a single female rat.

### BRIEF DESCRIPTION OF DRAWINGS

FIG. 1: The figure shows the results of applying the method of the present invention to 5-week-old Crl:CD (SD) female rats. Data represent mean±SEM, and each point represents an individual subject (rat). * indicates P<0.05.

FIG. 2: The figure shows a photograph of fertilized eggs derived from superovulation-induced oocytes, which were obtained by inducing superovulation by the method of the present invention and subjected to in vitro fertilization with fresh sperm of the same rat strain, Crl:CD (SD).

FIG. 3: The figure shows a photograph of offspring produced by transplanting fertilized eggs, which were produced by in vitro fertilization between oocytes obtained by inducing superovulation using the method of the present invention and fresh sperm, into the oviduct of a surrogate mother.

FIG. 4: The figure shows a photograph of offspring produced by transplanting fertilized eggs, which were produced by in vitro fertilization between oocytes obtained by inducing superovulation using the method of the present invention and frozen-thawed sperm of EGFP transgenic rats, into the oviduct of a surrogate mother.

FIG. 5: The figure shows the results of applying the method of the present invention to 5-week-old female Wistar rats. Data represent mean±SEM, and each point represents an individual subject (rat) (the same applies to the following figures unless otherwise specified). * indicates P<0.05, and n.s. indicates “not significant” (the same applies to the following figures unless otherwise specified).

FIG. 6: The figure shows the results of applying the method of the present invention to 5-week-old female LE rats.

FIG. 7: The figure shows the results of applying the method of the present invention to 5-week-old female F344 rats.

FIG. 8: The figure shows the results of applying the method of the present invention to 5-week-old female BN rats.

FIG. 9: The figure shows the results of applying the method of the present invention to 5-week-old female LEW rats.

FIG. 10: The figure shows the results of applying the method of the present invention to 3-week-old female Crl:CD (SD) rats.

FIG. 11: The figure shows the results of applying the method of the present invention to 6-week-old female Crl:CD (SD) rats.

FIG. 12: The figure shows the results of applying the method of the present invention to 7-week-old female Crl:CD (SD) rats.

### DESCRIPTION OF EMBODIMENTS

Hereinafter, the present invention will be described, by way of example, with preferred methods and materials that can be used in the practice of the present invention, but the present invention is not limited to the embodiments described below. Unless otherwise specified in the text, all technical and scientific terms used herein have the same meaning as commonly understood by those skilled in the art to which the present invention pertains. Any materials and methods similar or equivalent to those described herein can also be used in the practice of the present invention. All publications and patents cited herein in connection with the present invention are incorporated herein by reference and constitute a part of this specification, for example, as showing methods, materials, and the like that can be used in the present invention.

In this specification, the expression “A to B” indicating a numerical range means a numerical range including the endpoints A and B. The same applies to “A through B.” In this specification, the term “about” is used in the sense of allowing a tolerance of 10%.

In this specification, pregnant mare serum gonadotropin (PMSG) and equine chorionic gonadotropin (eCG) refer to the same substance and are used as interchangeable terms. Also, in this specification, anti-inhibin serum (AIS) and inhibin antiserum (IAS) refer to the same substance and are used as interchangeable terms.

The present inventors have found that simultaneous administration of inhibin antiserum and equine chorionic gonadotropin (eCG) to female rats less than 6 weeks of age, followed by administration of human chorionic gonadotropin (hCG), can efficiently induce superovulation. In one aspect, the present invention provides a method for inducing superovulation, comprising simultaneous administration of inhibin antiserum and equine chorionic gonadotropin (eCG) to female rats under 6 weeks of age, preferably between 3 and 6 weeks of age, followed by administration of human chorionic gonadotropin (hCG).

Another aspect of the present invention is an in vitro fertilization (IVF) method comprising the steps of: (i) administering inhibin antiserum and equine chorionic gonadotropin (eCG) simultaneously to female rats less than 6 weeks of age, preferably between 3 and 6 weeks of age, and then administering human chorionic gonadotropin (hCG) to induce superovulation; (ii) collecting oocytes from the female rats in which superovulation has been induced; and (iii) fertilizing the collected oocytes with sperm from a male rat, preferably sperm from a genetically modified male rat.

The female rats used in the method of the present invention may be of any strain. Examples thereof include the strain of female rats from Jackson Laboratory and the strain of female rats from Japan SLC, Inc. Examples of female rat strains include, but are not limited to, Crl:CD (SD), Crlj:WI, Crlj:LE, F344/DuCrlCrlj, LEW/CrlCrlj, BN/CrlCrlj, WKY/NCrlCrlj, Slc:SD, Slc:Wistar, Slc:Wistar/ST, lar:Long-Evans (LE), F344/NSlc, DA/Slc, LEW/SsNSlc, BN/ScNSlc, and ACI/NSlc, with SD, Wistar, F344, LEW, LE, and BN strains, frequently used, being particularly preferred. Genetically modified rats (transgenic rats) and various disease model rats produced using these strains can also be used in the methods of the present invention. Examples of disease model rats include, but are not limited to, PCK/CrlCrlj-Pkhd1^{pck}/CrlCrlj, ZDF-Lepr^{fa}/CrlCrlj, GK/Slc, Slc:Zucker-fa/fa, Hos:ZFDM-Lepr^{fa}, SHR/lzm, SHRSP/lzm, DIS/EisSlc, DIR/EisSlc, SHRAP5/Dmcr, and HWY/Slc. Furthermore, new strains of female rats, genetically modified rats, and disease model rats that will be established in the future can also be used in the methods of the present invention.

Female rats reach sexual maturity between 50 and 80 days, and rats younger than this age are called immature rats. Therefore, rats 7 weeks or younger are considered immature, but the method of the present invention is characterized by using younger female rats less than 6 weeks of age. The age of the female rats used in the present invention is between 3 and 6 weeks, preferably between 3 and 5 weeks, and more preferably between 4 and 5 weeks. In this specification, 3 weeks of age refers to 21 to 27 days after birth, 4 weeks of age refers to 28 to 34 days after birth, and 5 weeks of age refers to 35 to 41 days after birth. Therefore, female rats aged between 3 and 6 weeks refer to female rats that are between 21 and 41 days after birth.

In the present invention, the term “age in weeks” refers to the age in weeks at the time of administration of PMSG (eCG), IAS or hCG, and preferably refers to the age in weeks at the time of administration of PMSG.

The anti-inhibin antibody used in the present invention may be any antibody that has the activity of neutralizing endogenous inhibin in rats. For example, an inhibin antiserum containing anti-inhibin antibodies can be produced by immunizing a castrated goat with purified 32 kDa pig, mouse, or rat inhibin as an antigen. Anti-inhibin activity can be measured by conventional methods. For example, immunological titer can be measured by immunoassay. In the present invention, the anti-inhibin antibody may be in the form of an inhibin antiserum or a purified (including crudely purified) antibody.

When an inhibin antiserum is used, for example, but not limited to, 0.05 mL to 0.3 mL, preferably 0.05 mL to 0.2 mL, more preferably 0.05 mL to 0.15 mL, and still more preferably about 0.1 mL of the prepared antiserum is administered to a rat.

The amount of equine chorionic gonadotropin (eCG) administered to female rats in the method for inducing superovulation of the present invention is the amount typically used in superovulation treatments in laboratory rats. For example, typically, the amount is 2 to 50 IU, preferably 5 to 40 IU, more preferably 10 to 30 IU, and still more preferably 15 to 25 IU per rat. For example, excellent superovulation induction can be achieved by administering 5 to 40 IU, preferably 10 to 30 IU of eCG per rat in combination with 0.05 to 0.3 mL, preferably 0.05 to 0.2 mL of IAS to female rats aged 3 to less than 6 weeks, more preferably 4 to 5 weeks.

The amount of human chorionic gonadotropin (hCG) administered to female rats in the method for inducing superovulation of the present invention is the amount typically used in superovulation treatments of laboratory rats, for example, 2 to 50 IU, preferably 5 to 40 IU, more preferably 10 to 30 IU, and still more preferably 15 to 25 IU per rat.

A feature of the method for inducing superovulation of the present invention is that an anti-inhibin antibody (e.g., inhibin antiserum) and equine chorionic gonadotropin (eCG) are simultaneously administered to female rats less than 6 weeks of age, followed by administration of human chorionic gonadotropin (hCG). The expression “simultaneous administrating” the anti-inhibin antibody and eCG means that they may be administered separately at the same time or together as a mixture, and in the case of separate administration, they only need to be administered at approximately the same time. The administration may be performed, for example, but not limited to, by intraperitoneal injection. After a certain period of time, for example, about 2 to 3 days, preferably about 2 days, hCG is then administered. The administration of hCG may be performed, for example, but not limited to, by intraperitoneal injection.

In the method for inducing superovulation of the present invention, treated female rats are less than 6 weeks of age, particularly between 3 and 6 weeks of age, and an anti-inhibin antibody (preferably an inhibin antiserum) and equine chorionic gonadotropin (eCG) are administered simultaneously, followed by administration of human chorionic gonadotropin (hCG), thereby enabling the production of significantly more oocytes than those obtained by previously reported methods.

The oocytes obtained by the present invention can be used by being fertilized with sperm from a male rat. The sperm can be collected from an adult male rat by a conventional method, and either in a fresh state or after refrigerated storage or frozen storage, be fertilized with the oocytes produced by the present invention. The male rat providing the sperm is preferably of the same strain as the female rat used for fertilization. The fertilization can be carried out by conventional methods. For example, it can be performed according to the reported methods (Non-Patent Documents 3-5: T. Aoto et al., Transgenic Res. (2011) 20:1245-1252, N Nakagata et al., Sci Rep. 2020 Jan. 9; 10(1):93. doi: 10.1038/s41598-019-57090-7, K. Yamaga et al., Sci Rep. 2021; 11: 22641. doi: 10.1038/s41598-021-02166-6, which are incorporated herein by reference). Fertilized eggs obtained from oocytes produced by the present invention can develop well into cleavage-stage embryos. This method of producing cleavage-stage embryos is also included in the present invention. Furthermore, the cleaved-stage embryos thus obtained can be transferred to a recipient by embryo transfer to produce offspring. Embryo transfer can be carried out by conventional methods. This method for producing rats is also included in the present invention.

Therefore, one aspect of the present invention is an in vitro fertilization method in which oocytes collected from a female rat by the method of inducing superovulation of the present invention are fertilized in vitro with sperm from a male rat (preferably genetically modified rats).

Another aspect of the present invention is a method for preparing cleavage-stage embryos by culturing fertilized eggs obtained by the in vitro fertilization method of the present invention.

Another aspect of the present invention is a method for producing offspring by transferring a cleaved-stage embryo obtained by the method for preparing a cleaved-stage embryo of the present invention into a recipient by embryo transfer.

As used herein, the term “exogenous” in exogenous gonadotropin-releasing hormone or luteinizing hormone-releasing hormone means a hormone that is not produced in the body of a living rat, but is administered to the rat from the outside. The exogenous gonadotropin-releasing hormone or luteinizing hormone-releasing hormone may be derived from a rat or another species (e.g., pigs or mice).

### EXAMPLES

The present invention will be specifically described below with reference to examples, but the present invention is not limited to the following examples.

### 1. Materials and Methods

### (1-1) Animals

Female and male rats of each strain were purchased from Jackson Laboratory Japan (formerly Charles River Japan). Female rats of the indicated ages were used as oocyte donors, and male rats at 12-14 weeks of age were used as sperm donors. All animals were housed with food and water ad libitum and under a 12-hour dark-light cycle (light hours: 7:00-19:00) at 22 °C±1 °C. Animal experiments were performed according to protocols approved by the Animal Care and Use Committee of Kumamoto University.

### (1-2) Culture Medium

Modified human tubal fluid (mHTF) supplemented with increased calcium was used as the preincubation medium for sperm and as the in vitro fertilization medium. Rat embryo culture medium (R1ECM) was used for manipulation of 2-cell embryos or for culture up to the blastocyst stage.

### (1-3) Ovulation and Collection of Oocytes

Female rats were administered PMSG (Aska Pharmaceutical Co., Ltd.) alone, a combination of IAS and PMSG, or a combination of LH-RH (Peptide Institute, Inc.), IAS and PMSG. IAS was prepared using mouse inhibin peptide according to the method described in the paper by Kishi et al. (Non-Patent Document 6: H. Kishi et al., The Journal of endocrinology 151(1): 65-75, 1996, which is incorporated herein by reference). The prepared antiserum was used as is. The antibody activity of the prepared IAS was measured using ELISA and standardized for use as necessary. Specifically, mouse inhibin peptide at a concentration of 5 g/mL was adsorbed onto an ELISA plate, followed by washing and blocking. Inhibin antiserum diluted 1,000-128,000 times was applied, and ELISA was performed using horseradish peroxidase-labeled anti-goat IgG antibody as a secondary antibody. It was confirmed that inhibin antibody could be sufficiently detected even at the maximum dilution, and the antiserum was used in the following experiments.

In the experimental group in which LH-RH was administered, LH-RH was administered about 2 days before the administration of PMSG. In the experimental group in which IAS was administered, IAS was administered simultaneously with PMSG.

### (1-4) In Vitro Fertilization

Sperm were collected from genetically modified rats of SD, Wistar, or LE strain backgrounds as follows. After male rats were euthanized by cervical dislocation, the cauda epididymis was collected and transferred to a sperm preculture dish covered with paraffin oil. Sperm masses were collected from the cauda epididymis using a glass rod and transferred into a 400 μL drop of mHTF medium, followed by secondary dilution into a 200 μL drop of mHTF medium to a final sperm concentration of 500 sperm/μL. For oocytes, approximately two days after PMSG administration, the rats were administered hCG (Aska Pharmaceutical Co., Ltd.). One day after the administration of hCG, the rats were euthanized by cervical dislocation, and the oviducts were quickly removed. Cumulus-oocyte complexes (COCs) were collected from the oviducts under microscopic observation and introduced into a 200 μL drop of mHTF medium containing sperms (insemination). Seven hours after insemination, the oocytes were washed in four drops (80 μL) of mHTF. After washing the oocytes, the number of oocytes was counted. At 22-24 hours after fertilization, the fertilization rate was calculated by dividing the total number of pronuclear-stage fertilized eggs and two-cell embryos by the total number of oocytes and multiplying by 100.

### (1-6) Embryo Transfer

Embryo transfer was performed according to the procedure previously reported by the present inventor (Non-Patent Document 7, Nakagata, N. Embryo transfer through the wall of the fallopian tube in mice. Exp. Anim. (Experimental Animals) 41(3), 387-388 (1992), which is incorporated herein by reference). In vitro fertilized eggs and two-cell embryos were transferred into the oviducts of CD (SD) female rats (10 embryos/oviduct) on the day when a vaginal plug was observed (day 1 of pseudopregnancy). The embryos were transferred through the oviduct wall. The number of offspring was recorded 22 days later.

### (1-7) Statistical Analysis

Statistical analysis was performed using Prism version 5.0 (GraphPad). Results are expressed as mean±standard deviation. Group results were compared using analysis of variance after arcsine transformation of the percentages. p<0.05 was considered statistically significant.

### Example 1

PMSG (eCG), IAS, and hCG were administered to 4-week-old or 5-week-old Crl:CD (SD) female rats under the following four conditions (1 to 4), and oocytes were obtained.

Condition 1: hCG (300 IU/kg) was administered 48 to 50 hours after administration of PMSG (300 IU/kg).

Condition 2: PMSG (300 IU/kg) was administered 52 to 53 hours after administration of LH-RH (0.04 mg, dissolved in 0.2 mL of physiological saline), and hCG (300 IU/kg) was administered 48 to 50 hours thereafter.

Condition 3: hCG (300 IU/kg) was administered 48 to 50 hours after administration of PMSG (300 IU/kg) and IAS (0.1 mL).

Condition 4: PMSG (300 IU/kg) and IAS (0.1 mL) were administered 52-53 hours after administration of LH-RH (0.04 mg, dissolved in 200 μL of physiological saline), and hCG (300 IU/kg) was further administered 48-50 hours thereafter.

Four female rats were used as oocytes donors in each group, and in vitro fertilization was performed between the obtained oocytes and sperm from genetically modified rats.

The results are shown in Table 1 below.

TABLE 1

| | Crl:CD(SD)(5 weeks of age) | | Crl:CD(SD)(4 weaks of age) | |
|---|---|---|---|---|
| | Mean number | number of fertilized | Mean number | number of fertilized |
| | of ovulated | oocytes/total | of ovulated | oocytes/total |
| | oocytes | number of oocytes | oocytes | number of oocytes |
| Condition 1: | 25.8 | 102/103(99.0%) | 40.8 | 153/163(93.9%) |
| PMSG → hCG | | | | |
| Condition 2: | 18.0 | 68/72(94.4%) | 39.5 | 146/158(92.4%) |
| LH-RH → PMSG → hCG | | | | |
| Condition 3: | 70.5 | 269/282(95.4%) | 81.0 | 287/324(88.6%) |
| IAS + PMSG → hCG | | | | |
| Condition 4: | 40.0 | 185/196(94.4%) | 72.3 | 253/289(87.5%) |
| LH-RH → IAS + PMSG → hCG | | | | |

It was found that simultaneous administration of IAS and PMSG to female rats, followed by administration of hCG, significantly induced superovulation. Furthermore, comparison between Condition 1 and Condition 2, and between Condition 3 and Condition 4, showed that there was no effect of LH-RH when 4-week-old and 5-week-old CD (SD) rats were used. In particular, in 5-week-old rats, it was found that administration of LH-RH suppressed the induction of superovulation by administration of IAS and PMSG. The fertilization rate was good in all cases.

### Example 2

The effects of LH-RH, IAS, and PMSG were examined using 5-week-old Crl:CD (SD) female rats under the following three conditions (experimental groups) in the same manner as in Example 1. The doses of LH-RH, IAS, PMSG, and hCG were 0.04 mg, 0.1 mL, 20 IU, and 20 IU per rat, respectively. The administration conditions were as follows:

Experimental group LH-RH: Two days after the administration of LH-RH, IAS and PMSG were administered, and two days thereafter, hCG was administered.

Experimental group IASe: IAS and PMSG were administered, and then hCG was administered two days thereafter.

Experimental group PMSG: PMSG was administered, and then hCG was administered two days thereafter.

In the same manner as in Example 1, in vitro fertilization was carried out using the obtained oocytes, and the fertilized eggs were then transplanted into recipients to produce offspring.

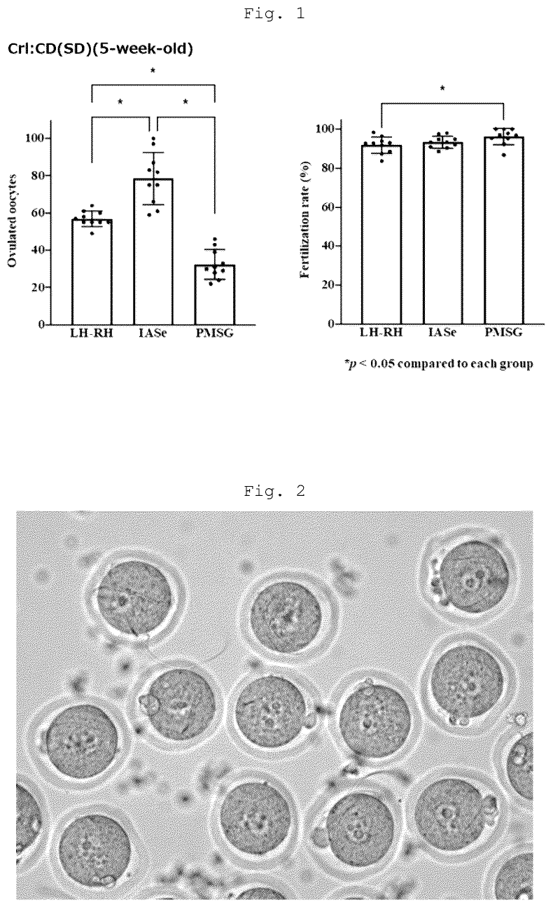

The results of the number of ovulated oocytes and fertility rate are shown in the table below and in FIG. 1.

TABLE 2

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 569 | 56.9 ± 4.7 | 525 | 92.3 ± 4.7^{a} |
| IASe | 0 | 0.1 | 20 | 10 | 785 | 78.5 ± 14.0 | 735 | 93.6 ± 3.0^{a, b} |
| PMSG | 0 | 0 | 20 | 10 | 325 | 32.5 ± 7.9 | 312 | 96.0 ± 4.1^{b} |

Different letters indicate significant differences (p < 0.05)

It was found that simultaneous administration of IAS and PMSG, followed by administration of hCG to 5-week-old Crl:CD(SD) female rats significantly induced superovulation. It was found that the effect of superovulation induction was also in the order of IASe>LH-RH>PMSG, and that administration of LH-RH suppressed the effect of superovulation induced by administration of IAS and PMSG. The fertilization rate was good in all cases.

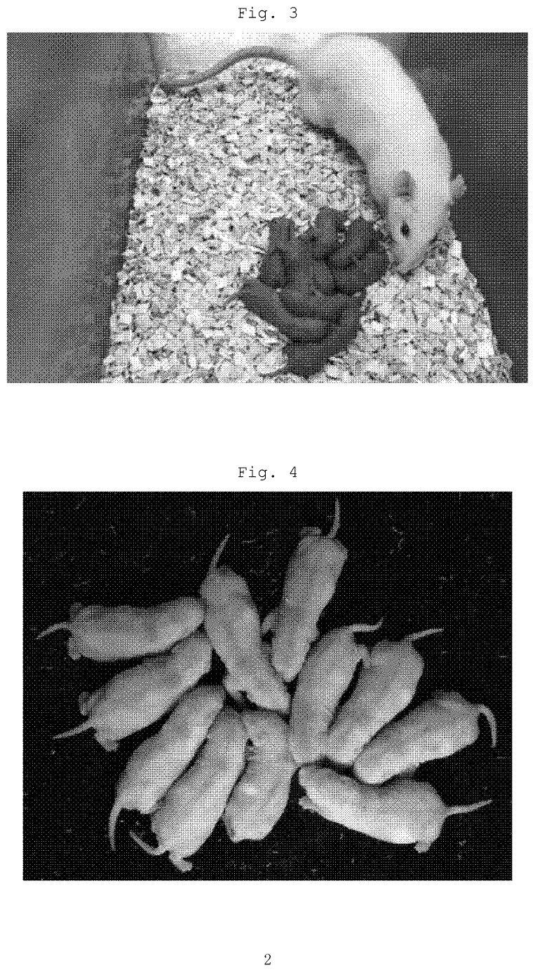

Using oocytes collected from superovulated female rats, in vitro fertilization was performed between the oocytes and fresh sperm from Crl:CD (SD) rats. A photograph of the resulting fertilized eggs is shown in FIG. 2. Pronuclei and sperm tails were observed within the ooplasm in all of the oocytes. Furthermore, a photograph of offspring produced by transplanting the fertilized eggs into the oviducts of a surrogate mother is shown in FIG. 3.

Furthermore, in vitro fertilization was performed between the oocytes collected from superovulated female rats and frozen-thawed sperm from EGFP (Enhanced Green Fluorescent Protein) transgenic rats, and the resulting fertilized eggs were transplanted into the oviducts of surrogate mothers to produce offspring. The results are shown in FIG. 4. Offspring were obtained that expressed the EGFP-derived gene throughout the body and exhibited green fluorescence. All of the obtained offspring expressed EGFP.

### Example 3

The effect of combined administration of IAS and PMSG on various strains of rats was investigated. In the same manner as in Example 2, the induction of superovulation by LH-RH, IAS, and PMSG was examined using 5-week-old female rats of various strains (Wistar, LE, F344, BN, and LEW).

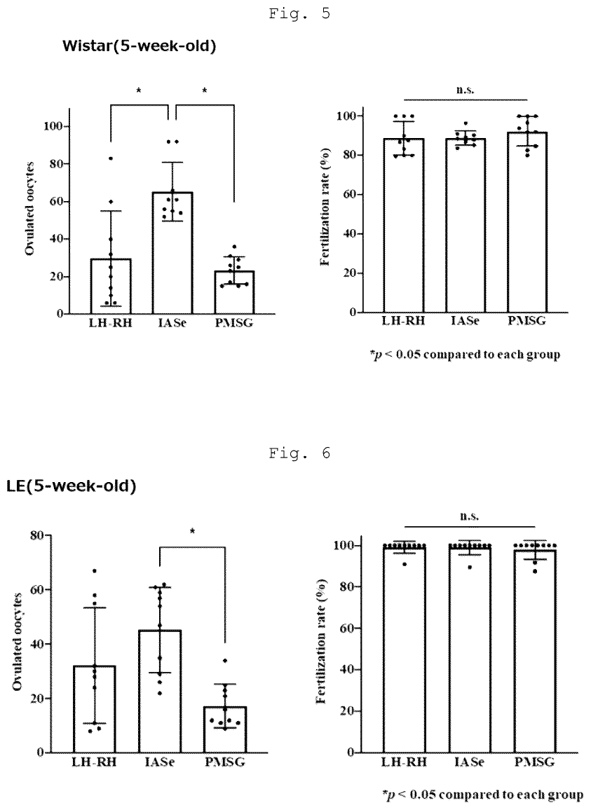

The results for the Wistar female rats are shown in the table below and in FIG. 5.

TABLE 3
Wistar (5-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 296 | 29.6 ± 25.3^{a} | 253 | 85.5 ± 8.5 |
| IASe | 0 | 0.1 | 20 | 10 | 589 | 65.4 ± 15.7^{b} | 521 | 88.5 ± 3.7 |
| PMSG | 0 | 0 | 20 | 10 | 233 | 23.3 ± 7.4^{a, c} | 215 | 92.3 ± 7.5 |

Different letters indicate significant differences (p < 0.05)

It was found that simultaneous administration of IAS and PMSG, followed by administration of hCG to 5-week-old Wistar female rats significantly induced superovulation. It was found that the effect of superovulation induction was also in the order IASe>LH-RH>PMSG, and that administration of LH-RH suppressed the effect of superovulation induced by administration of IAS and PMSG. The fertilization rate was good in all cases.

The results for LE female rats are shown in the table below and in FIG. 6.

TABLE 4
LE (5-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 322 | 32.2 ± 21.2^{a, b} | 317 | 98.4 ± 2.9 |
| IASe | 0 | 0.1 | 20 | 10 | 452 | 45.2 ± 15.7^{a} | 446 | 98.7 ± 3.3 |
| PMSG | 0 | 0 | 20 | 10 | 174 | 17.4 ± 8.1^{b} | 171 | 98.3 ± 4.5 |

Different letters indicate significant differences (p < 0.05)

It was found that simultaneous administration of IAS and PMSG, followed by administration of hCG to 5-week-old LE female rats significantly induced superovulation. It was found that the effect of superovulation induction was also in the order of IASe≈LH-RH>PMSG, and that administration of LH-RH tended to suppress the effect of superovulation induced by administration of IAS and PMSG. The fertilization rate was good in all cases.

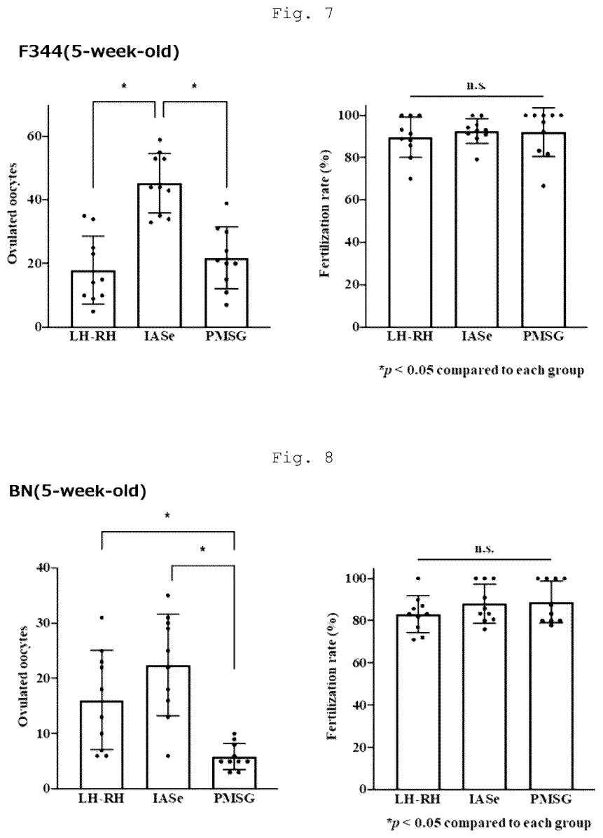

The results for F344 female rats are shown in the table below and in FIG. 7.

TABLE 5
F344 (5-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 180 | 18.0 ± 10.7^{a} | 164 | 91.1 ± 9.6 |
| IASe | 0 | 0.1 | 20 | 10 | 453 | 45.3 ± 9.4^{b} | 418 | 92.3 ± 5.9 |
| PMSG | 0 | 0 | 20 | 10 | 218 | 21.8 ± 9.7^{a, c} | 200 | 91.7 ± 11.4 |

Different letters indicate significant differences (p < 0.05)

It was found that simultaneous administration of IAS and PMSG, followed by administration of hCG to 5-week-old F344 female rats significantly induced superovulation. It was found that the effect of superovulation induction was also in the order IASe>LH-RH>PMSG, and that administration of LH-RH suppressed the effect of superovulation induced by administration of IAS and PMSG. The fertilization rate was good in all cases.

The results for BN female rats are shown in the table below and in FIG. 8.

TABLE 6
BN (5-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 161 | 16.1 ± 8.9^{a} | 129 | 80.1 ± 8.6 |
| IASe | 0 | 0.1 | 20 | 10 | 225 | 22.5 ± 9.2^{a} | 192 | 85.3 ± 9.2 |
| PMSG | 0 | 0 | 20 | 10 | 59 | 5.9 ± 2.4^{b} | 51 | 86.4 ± 9.9 |

Different letters indicate significant differences (p < 0.05)

It was found that simultaneous administration of IAS and PMSG, followed by administration of hCG to 5-week-old BN female rats significantly induced superovulation. It was found that the effect of superovulation induction was also in the order of IASe≈LH-RH>PMSG, and that administration of LH-RH tended to suppress the effect of superovulation induced by administration of IAS and PMSG. The fertilization rate was good in all cases.

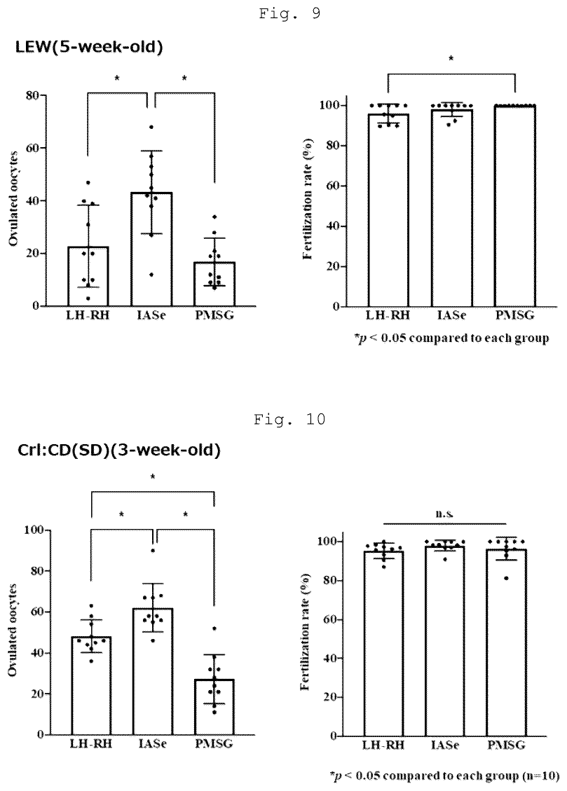

The results for LEW female rats are shown in the table below and in FIG. 9.

TABLE 7
LEW (5-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 228 | 22.8 ± 15.5^{a} | 216 | 94.7 ± 4.6^{a} |
| IASe | 0 | 0.1 | 20 | 10 | 433 | 43.3 ± 15.7^{b} | 424 | 97.9 ± 3.6^{a, b} |
| PMSG | 0 | 0 | 20 | 10 | 169 | 16.9 ± 9.0^{a, c} | 169 | 100.0 ± 0.0^{b} |

Different letters indicate significant differences (p < 0.05)

It was found that simultaneous administration of IAS and PMSG, followed by administration of hCG to 5-week-old LEW female rats significantly induced superovulation. It was found that the effect of superovulation induction was also in the order IASe>LH-RH>PMSG, and that administration of LH-RH suppressed the effect of superovulation induced by administration of IAS and PMSG. The fertilization rate was good in all cases.

### Example 4

The effects of IAS and PMSG were examined using 3-week-old Crl:CD (SD) female rats in the same manner as in Example 2. The doses of IAS, PMSG, and hCG were 0.1 mL, 20 IU, and 20 IU per rat, respectively. The administration conditions were as follows: IAS and PMSG were administered, followed by hCG two days thereafter. In vitro fertilization was performed using the resulting oocytes in the same manner as in Example 2. The results are shown in the table below and in FIG. 10. Even at 3 weeks of age, the ovulation effect was confirmed to be almost equivalent to that at 4 and 5 weeks of age.

TABLE 8
Crl: CD(SD) (3-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 10 | 482 | 48.2 ± 8.0^{a} | 460 | 95.4 ± 4.0 |
| IASe | 0 | 0.1 | 20 | 10 | 621 | 62.1 ± 11.9^{b} | 609 | 98.1 ± 2.8 |
| PMSG | 0 | 0 | 20 | 10 | 273 | 27.3 ± 12.0^{c} | 261 | 95.6 ± 5.9 |

Different letters indicate significant differences (p < 0.05)

### (Comparative Example) Effect of LH-RH on Superovulation Induction by IAS+PMSG

According to the report by Honda et al. (Non-Patent Document 2), when immature BN rats were administered LH-RH, followed by simultaneous administration of IAS and PMSG, and then administration of hCG, ovulation was induced more significantly compared with cases in which LH-RH was not administered. Similar trends have also been shown to be observed in other strains of rats (Wistar, F344, LE, and SD).

However, as shown in Examples 1 to 3, it has been found that administration of IAS and PMSG significantly induces superovulation in all strains of rats, but the superovulation-inducing effect of administration of IAS and PMSG is suppressed by administration of LH-RH. Furthermore, this suppressive effect was more pronounced at 5 weeks of age than at 4 weeks of age in Crl:CD(SD) female rats.

To confirm this difference, the effects of LH-RH, IAS, and PMSG on the induction of superovulation were examined using 6-week-old and 7-week-old immature female rats.

In the same manner as in Example 2, the effects of LH-RH, IAS, and PMSG were examined under the following three conditions (experimental groups) using 6-week-old and 7-week-old Crl:CD (SD) female rats. The doses of LH-RH, IAS, PMSG, and hCG were 0.04 mg, 0.1 mL, 20 IU, and 20 IU per rat, respectively. The administration conditions were as follows:

Experimental group LH-RH: Two days after the administration of LH-RH, IAS and PMSG were administered, and two days thereafter, hCG was administered.

Experimental group IASe: IAS and PMSG were administered, and then hCG was administered two days thereafter.

Experimental group PMSG: PMSG was administered, and then hCG was administered two days thereafter.

In the same manner as in Example 1, in vitro fertilization was carried out using the obtained oocytes.

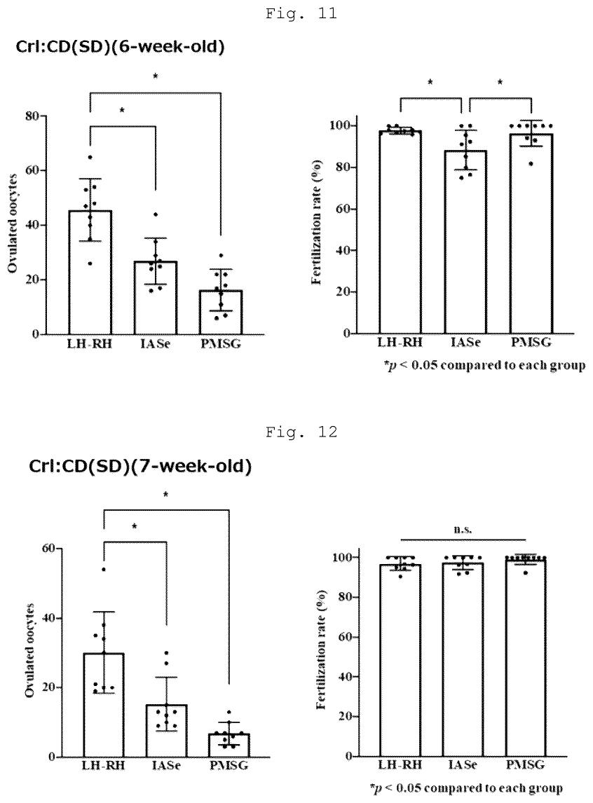

The results of the number of ovulated oocytes and the fertilization rate are shown in the following tables and figures. Table 9 and FIG. 11 show the results for 6-week-old rats, and Table 10 and FIG. 12 show the results for 7-week-old rats.

TABLE 9
Crl: CD(SD) (6-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 9 | 411 | 45.7 ± 11.4^{a} | 401 | 97.5 ± 1.5^{a} |
| IASe | 0 | 0.1 | 20 | 9 | 242 | 26.9 ± 8.5^{b} | 218 | 90.1 ± 9.6^{a} |
| PMSG | 0 | 0 | 20 | 9 | 147 | 16.3 ± 7.5^{b} | 140 | 95.2 ± 6.2^{a} |

Different letters indicate significant differences (p < 0.05)

When 6-week-old Crl:CD (SD) female rats were simultaneously administered IAS and PMSG, followed by administration of hCG, a tendency toward superovulation was observed, but no statistically significant effect was observed compared to PMSG alone. However, administration of LH-RH resulted in a significant effect of inducing superovulation. This result was consistent with the report by Honda et al.

TABLE 10
Crl: CD(SD) (7-week-old)

| | | | | | Total | | Number | |
|---|---|---|---|---|---|---|---|---|
| | | | | Number of | number of | | of | |
| Exp. | LH-RH | IAS | PMSG | donar rats | ovulated | Mean number of | fertilized | Fertilization rate |
| Group | (g) | (mL) | (IU) | (for ovulation) | oocytes | ovulated oocytes | oocytes | (%) |
| LH-RH | 0.04 | 0.1 | 20 | 9 | 271 | 31.1 ± 11.6^{a} | 263 | 97.0 ± 3.4 |
| IASe | 0 | 0.1 | 20 | 9 | 138 | 15.3 ± 7.8^{b} | 134 | 97.1 ± 3.4 |
| PMSG | 0 | 0 | 20 | 9 | 61 | 6.8 ± 3.2^{b} | 60 | 98.4 ± 2.6 |

Different letters indicate significant differences (p < 0.05)

When 7-week-old Crl:CD (SD) female rats were simultaneously administered IAS and PMSG, followed by administration of hCG, a tendency toward superovulation was observed, but no statistically significant effect was observed compared to PMSG alone. However, administration of LH-RH resulted in a significant effect of inducing superovulation. This result was consistent with the report by Honda et al.

The above results indicate that the effect of LH-RH administration on immature rats differs between 6-week-old or 7-week-old rats and younger 4-week-old or 5-week-old rats. Specifically, the superovulation-inducing effect of IAS and PMSG administration can be observed in 6-week-old and 7-week-old rats, but it is not very pronounced, whereas a significantly effect is observed in 4-week-old and 5-week-old rats. Furthermore, the superovulation-inducing effect of IAS and PMSG administration is significantly amplified by LH-RH administration in 6-week-old and 7-week-old rats, whereas it is suppressed in 4-week-old and 5-week-old rats. Thus, both the superovulation-inducing effect of IAS and PMSG administration and the effect of LH-RH thereon differ depending on the age of the rats used.

The foregoing detailed description is merely intended to illustrate the objects and scope of the present invention and is not intended to limit the scope of the appended claims. Various modifications and substitutions to the described embodiments will be apparent to those skilled in the art from the teachings set forth herein, without departing from the scope of the appended claims.

### INDUSTRIAL APPLICABILITY

The present invention provides a method for inducing superovulation in rats. The method of the present invention can reduce the number of female rats used as oocyte donors, and is extremely useful for rat resource banks that can strongly promote the realization of the 3Rs principle in animal experiments and research using genetically modified rats, and for improving the efficiency of research in research laboratories around the world.

This application is based on Japanese patent application No. 2023-070207 filed in Japan (filing date: Apr. 21, 2023), the entire contents of which are incorporated into this specification.

1. A method for inducing superovulation, comprising simultaneously administering an anti-inhibin antibody and equine chorionic gonadotropin (eCG) to a female rat less than 6 weeks of age, and subsequently administering human chorionic gonadotropin (hCG).

2. The method according to claim 1, wherein the anti-inhibin antibody is administered as inhibin antiserum.

3. The method according to claim 2, wherein the female rat is 3 weeks of age or older.

4. The method according to claim 2, wherein the female rat is 3 to 5 weeks of age.

5. The method according to claim 2, wherein the female rat has not been administered exogenous luteinizing hormone-releasing hormone (LH-RH) prior to administration of equine chorionic gonadotropin.

6. The method according to claim 2, wherein the female rat has not been administered exogenous gonadotropin-releasing hormone prior to administration of equine chorionic gonadotropin.

7. The method according to claim 2, wherein the inhibin antiserum to be administered is 0.3 mL or less per rat.

8. The method according to claim 7, wherein the inhibin antiserum to be administered is 0.05 mL or more and 0.2 mL or less per rat.

9. The method according to claim 8, wherein the equine chorionic gonadotropin to be administered is 5 IU or more and 40 IU or less per rat.

10. The method according to claim 1, wherein the female rat is of a strain selected from the group consisting of CD (SD), Wistar, Long-Evans, F344, BN and LEW.

11. An in vitro fertilization method comprising the steps of:
a. producing oocytes using the method according to claim 1; and
b. fertilizing the oocytes produced in the step (a) with sperm from a male rat.

12. An in vitro fertilization method comprising the steps of:
(1) inducing superovulation by simultaneously administering an anti-inhibin antibody and equine chorionic gonadotropin (eCG) to a female rat less than 6 weeks of age, and subsequently administering human chorionic gonadotropin (hCG);
(2) collecting oocytes from the superovulated female rat; and
(3) fertilizing the oocytes collected in the step (2) with sperm from a male rat.

13. The method according to claim 12, wherein the anti-inhibin antibody is administered as inhibin antiserum.

14. The in vitro fertilization method according to claim 13, wherein the sperm is derived from a male rat of a strain selected from the group consisting of CD (SD), Wistar, Long-Evans, F344, BN and LEW.

15. The method according to claim 14, wherein the female rat is 3 to 5 weeks of age.

16. The method according to claim 14, wherein the female rat has not been administered exogenous luteinizing hormone-releasing hormone (LH-RH) prior to administration of equine chorionic gonadotropin.

17. The method according to claim 13, wherein the inhibin antiserum to be administrated is 0.3 mL or less.

18. The method according to claim 17, wherein the inhibin antiserum to be administrated is 0.05 mL or more and 0.2 mL or less.

19. The method according to claim 18, wherein the equine chorionic gonadotropin to be administrated is 5 IU or more and 40 IU or less.
